(19) United States
(12) Patent Application Publication
(10) Pub. No.: US 2017/0246403 A1
(43) Pub. Date: Aug. 31, 2017

(54) AUTOMATIC INJECTION DEVICES

(71) Applicant: OWEN MUMFORD LIMITED, Oxford (GB)

(72) Inventors: Toby COWE, Oxfordshire (GB); Matthew FARMER, Oxfordshire (GB); Tim MULLER, Oxfordshire (GB)

(21) Appl. No.: 15/514,721

(22) PCT Filed: Sep. 30, 2015
(86) PCT No.: PCT/GB2015/052852

§ 371 Date: Mar. 27, 2017

(30) Foreign Application Priority Data
Sep. 30, 2014 (GB) 1417285.2
Oct. 3, 2014 (GB) 1417522.8
Apr. 30, 2015 (GB) 1507499.0

(51) Int. Cl. A61M 5/32; A61M 5/315; A61M 5/50; A61M 5/20

(52) U.S. Cl. CPC A61M 5/3257; A61M 5/2033; A61M 5/3202; A61M 5/31511; A61M 5/5086; A61M 2005/2013; A61M 2005/206; A61M 2005/2073; A61M 2005/208

(57) ABSTRACT

An injection device for delivering a dose of medicament from a syringe includes: a housing including a forward body and rearward body and wherein the forward and rearward body are axially moveably interconnected; a drive mechanism arranged, in use, to be released from an energized position to deliver a dose from the syringe; a latch arranged to hold the drive mechanism in the energized position; and a trigger for releasing the latch; wherein the injection device further includes an interlock arrangement moveable between a locked position in which release of the drive mechanism is prevented and an unlocked position in which the drive mechanism may be released, the interlock arrangement including: an internal blocking formation associated with the rearward body and positioned between the latch and trigger, and wherein in use, forward movement of the rearward body relative to the forward body moves the blocking formation into the unlocked position.

### FIELD OF THE INVENTION

This invention relates to injection devices for delivering a dose of medicament from a syringe. In particular, but not exclusively the invention relates to an “autoinjector” device.

### BACKGROUND OF THE INVENTION

Injection devices are used for the convenient administration of medicaments. For example, injection devices (which may typically be in the form of a pen injector) may be used for providing a single metered dose of a medicament, such as Epinephrine, in an emergency or for providing regular metered doses of a medicament, such as Insulin. Such devices may be either single use “disposable” devices in which the device is typically provided with a syringe already installed, and which is not user-replaceable, or “reusable” devices which allow the user to replace the syringe when the medicament has been used.

It may be noted that whilst the term “syringe” is used herein for clarity and consistency, however the skilled person will appreciate that this term is intended to be broadly interpreted. In some arrangements the syringe may for example be a cartridge (which, for example, may be arranged to receive a disposable needle) or other medicament container. In some arrangements the syringe/cartridge/medicament container may formed integrally with the (or part of the) injection device.

Injection devices may be provided in the form of an “autoinjector” device in which, in addition to automating the delivery of the medicament, the device is also arranged to automate the insertion of a needle into the skin prior to the delivery of the medicament.

Injection devices generally comprise a delivery mechanism which is arranged to automatically deliver a dose from the syringe, and optionally (in the case of an autoinjector) to first displace the syringe within the housing to cause needle penetration. The delivery mechanism is typically released from an energised (or primed) position and may, for example, include one or more drive springs. The delivery mechanism may act upon a plunger which includes or acts against a piston (also referred to as a “bung”) which is slidably provided within the syringe. In the case of an autoinjector the initial stiction between the piston and syringe may resist forward movement of the piston relative to the syringe such that initially the drive mechanism moves the syringe into the needle insertion position (whereupon further movement of the syringe is blocked and the drive mechanism will continue to move forward thus moving the piston).

A trigger, for example in the form of a button on the end of the device, is generally provided to allow the user to activate the delivery sequence. It is known to provide a safety arrangement in the form of a mechanical lock (which is referred to herein as an “interlock”) arranged to prevent actuation of the delivery mechanism by the user unless the lock is in the unlocked position. For example, such safety arrangements may require the user to perform a readying action prior to releasing the trigger or may be arranged to require the injection device to be in contact with an injection site before the trigger is able to activate the delivery mechanism. Examples of such arrangements are, for example, shown in: European Patent EP2429613, U.S. Pat. No. 5,137,516 and International Patent Application WO2012/110577.

The provision of safety mechanisms is desirable to avoid inadvertent operation of injection devices (particularly, in auto-injectors where inadvertent operation could result in injury due to the projection of the needle during the insertion stage of the activation sequence). However, such arrangements may result in increased complexity of the device. Increased complexity may for example result in less intuitive use of the device for the end user. Further increased complexity may increase manufacturing costs, by increasing part count and/or increasing assembly complexity, which may be particularly disadvantageous for disposable injection devices. Thus, it is desirable to provide an alternate injection device which may at least partially address some of these disadvantages.

### SUMMARY OF THE INVENTION

Accordingly, in one aspect, this invention provides an injection device for delivering a dose of medicament from a syringe, the injection device comprising:
a housing comprising a forward body and rearward body and wherein the forward and rearward body are axially moveably interconnected;
a drive mechanism arranged, in use, to be released from an energised position to deliver a dose from said syringe;
a latch arranged to hold the drive mechanism in the energised position; and
a trigger for releasing the latch; wherein
the injection device further comprises an interlock arrangement moveable between a locked position in which release of the drive mechanism is prevented and an unlocked position in which the drive mechanism may be released, the interlock arrangement comprising:
an internal blocking formation associated with the rearward body and positioned between the latch and trigger, and wherein
in use, forward movement of the rearward body relative to the forward body moves the blocking formation into the unlocked position.

Typically, the front and rear bodies of the housing are slidably interconnected. The front and rear bodies jointly define the housing which encloses the injection device. The front and rear bodies may be arranged to move relative to one another by a limited amount in the axial direction of the injector device.

The drive mechanism may further be arranged to displace the syringe within the housing to cause needle penetration in use. As such the injection device may further comprise a syringe carriage axially movable within the housing. For example the carriage may be moveable between a rearward (retracted) position and a forward (injection) position. The injection device may be an autoinjector.

The trigger and latch may remain axially fixed relative to one another when the interlock is moved to the unlocked position. As such, the spacing between the latch and trigger may remain constant during the interlock operation. Movement of the rearward body relative to the forward body may move the blocking formation relative to both the latch and trigger. The latch and trigger may both be fixed relative to the forward body during forward movement of the rearward body (to move the interlock to the unlocked position. Fixing both the latch and trigger relative to the forward body (in the initial interlock operation) will ensure that the latch and trigger remain relatively fixed.

In the locked position, the blocking formation may be arranged to block forward movement of the trigger. The blocking formation may, for example comprise a rearwardly facing surface, for example a shoulder. The rearwardly facing surface of the blocking member may be arranged for cooperating with a forwardly facing surface of the trigger when in the locked position. The forward facing surface may at least partially comprise a shoulder. The forward facing surface may at least partially comprise an end wall. A shoulder on the trigger may be formed on an inner surface of the trigger.

The surfaces of the blocking member and forward facing surface may have matching profiles to positive engage or align in the locked position. The cooperating surfaces may be substantially radially extending with respect to the injection device. The cooperating surfaces may be sloped or angled.

The latch may be arranged to expand in a radially outward direction to release the drive mechanism. At least a portion of the trigger may be disposed radially outside of the latch member. The trigger may prevent radial expansion of the latch in the pre-firing position. A surface of the trigger may abut an opposing surface to prevent radial movement of the latch member. When the trigger is in the release position the opposing surfaces of the trigger and latch may move out of axial alignment to enable the latch to expand.

In the locked position, the rearward body may also block release of the latch. For example, the rearward body may prevent expansion of the latch. The inner surface of the rearward body may be profiled such that the relative forward movement of the rearward body creates clearance between the body and the portion of the latch to allow radial expansion of the latch. For example the inner diameter of the rearward body could be outwardly tapered towards the rear of the body. Alternatively or additionally, the inner surface of the rearward body may comprise at least one inwardly extending abutment arranged to be moved axially into and out of blocking alignment with a portion of the latch.

The trigger may be disposed at a rearward end of the housing. The trigger may be arranged for forward movement relative to the housing to release the latch. The trigger may extend forwardly within the interior of the rearward body and the blocking formation may be at least partially within the interior of the trigger. Thus, the blocking formation may be at least partially captive within the trigger.

The latch may be at least partially enclosed by the trigger. For example, the latch and trigger may be substantially concentric. The trigger and latch may further be substantially concentric with the rearward body.

The trigger may extend forward to engage a feature of, or associated with, the forward body. The trigger may extend over a feature of, or associated with the rearward body to engage the feature of, or associated with, the forward body. The engagement between the trigger and forward body may be arranged to retain the forward and rearward bodies in slidable engagement.

This arrangement may be particularly advantageous in providing an injector device which can be easily assembled.

According to a further aspect the present invention comprises an injection device for delivering a dose of medicament from a syringe, the injection device comprising:
a housing comprising a forward body and rearward body and wherein the forward and rearward body are axially movably interconnected;
a drive mechanism arranged, in use, to be released from an energised position to deliver a dose from said syringe;
a trigger disposed at a rearward end of the housing for releasing the drive mechanism; wherein
trigger arranged to extend forwardly within the interior of the rearward body and over at least one feature of, or associated, with the rearward body and engages a feature of, or associated with, the forward body to retain the forward and rearward bodies in slidable engagement.

The axially movable interconnection between the forward and rearward body may be provided to enable relative movement during an activation sequence.

An axially extending substantially annular space may be defined between the trigger and feature of, or associated with, the forward body. The at least one feature of, or associated, with the rearward body over which the trigger extends may be captively received within said substantially annular space. Thus, the feature of, or associated with, the rearward body may be captive within the space but provided with a degree of axial freedom to allow relative axial movement between the forward body and rearward body in use.

The at least one feature of, or associated, with the rearward body may comprises a blocking formation of an interlock arrangement.

The trigger may comprise a cup-shaped button. The button may have a rearward end which substantially closes the rear end of the housing. The button may have side portions which extend forwardly within the interior of the rearward body.

The trigger may be arranged to have a snap fit engagement with the feature of, or associated with the forward body. The feature of, or associated with the forward body may be the latch (which may be connected to the forward body).

The trigger may include a resilient catch for engaging the feature of, or associated with the forward body (for example the latch). The resilient catch may be arranged to disengage, in use, to allow forward movement of the trigger. For example the catch or corresponding surface may have a ramped profile to allow disengagement in only the forward direction. The resilient catch may comprise at least one sprung finger extending forwardly from the trigger.

The resilient catch may be arranged to re-engage a feature of, or associated with the forward body (for example the latch) upon reaching an activation position. The resilient catch may re-engage at a second axial position relative to the forward body. Thus, the trigger may remain in an activation position after use to prevent re-activation and/or to provide a visual or tactile indication that the injector device has been activated.

The trigger may be arranged to be retained in two axial positions. A first, rearward, position may correspond to the unactivated position. A second, forward, position may correspond to the activated position. The trigger may be prevented from rearward movement in both axial positions. As such, prior to use the trigger may only be moved forward of the first axial position (towards the second axial position) and after reaching the second axial position the trigger may be prevented from returning towards the first axial position.

The rearward body may be arranged to prevent disengagement of the resilient catch until the rearward body is moved forward relative to the forward body. Such an arrangement provides a forced activation sequence whereby the injection device must be placed against the injection site before the trigger may be activated (in contrast to arrangements in which the two movements can be performed in either order or as a single motion). For example, the inner surface of the rearward body may be profiled such that the relative forward movement of the rearward body creates clearance between the resilient catch and the rearward body to allow disengagement of the resilient catch. For example the inner surface may include a tapered profile and/or may include an abutment surface which moves out of axial alignment with movement of the rearward body.

The trigger may engage a feature of the latch. Thus, the feature of, or associated with, the forward body may comprise the latch. The latch may engage the forward body. For example the latch and forward body may resiliently engage with a snap-fit arrangement. This arrangement may further simplify assembly of the device as the injector may be assembled into forward and rearward sub-assemblies (each within the respective body portion) before the syringe is positioned within the device and the sub-assemblies brought together such that the latch and forward body positively engage.

Thus, according to a further embodiment there is provided an injection device for delivering a dose of medicament from a syringe, the injection device comprising:
a housing comprising a forward body and rearward body and wherein the forward and rearward body are axially moveably interconnected;
a drive mechanism arranged, in use, to be released from an energised position to deliver a dose from said syringe;
a latch arranged to hold the drive mechanism in the energised position; and
a trigger for releasing the latch; wherein
the latch is arranged to provide an interconnecting member between the forward body and rearward body.

In an initial/pre-use condition the drive mechanism may be substantially contained within the rearward body. In an initial/pre-use condition the syringe may be substantially contained within the forward body. The forward body may contain a syringe carrier. The forward body may comprise or contain a moveable needle shroud.

The latch may be arranged to engage the forward body. The trigger may be arranged to engage the latch. A feature of, or associated with, the rearward body may be restrained between the latch and trigger.

The drive mechanism may comprise a plunger arranged for expelling medicament from the syringe. The plunger may be driven forwardly in use by the drive mechanism. The plunger may typically be arranged to engage a bung (or piston) within the syringe in use. The latch may engage a rearward portion of the plunger to hold the drive mechanism in the energised position.

The blocking formation may be co-moulded with the rearward body. The blocking formation may be generally radially spaced from the inner surface of the rearward body. It will be appreciated that such a radially spaced arrangement may by supported by a plurality of supports which extend inwardly from the inner wall of the rearward body. The blocking formation may comprise an internal wall section within the bore of the rearward body. The blocking formation may comprise a radial flange. The blocking formation may be substantially annular. The blocking formation may be at least partially annular. The blocking formation may be generally concentric with the rearward body. Whilst the blocking formation may be formed as a substantially continuous circumferential feature, it will also be appreciated that the blocking formation could comprise a plurality of discreet sections or segments (for example circumferentially spaced around the inner wall of the rearward body).

At least a portion of the internal wall extends generally axially relative to the rearward body. Such an axial portion may, for example, define a groove for retaining a spring. Alternatively, or additionally, an axial portion may be arranged to help align the rearward body and latch member.

The rearward body may be biased rearwardly relative to the forward body. As such, the interlock arrangement may be normally engaged. The rearward body may need to be urged forward against said bias to move the interlock to the unlocked position.

For example, the rearward and forward bodies are biased by a spring acting between the latch and blocking formation. The spring may be captive between the latch and blocking formation. The latch may comprise a rearwardly facing surface for engaging a first end of the spring. The blocking formation may comprise a forwardly facing surface for engaging a second end of the spring of the trigger. The spring may be a coil spring. Alternatively a moulded spring formation such as a spring arm may be provided acting between the body portions.

The rearward body may be arranged to be held by the user during use. Thus, the rearward body may move forwardly relative to the forward body when the injection device is pressed against an injection site.

Forward movement of the rearward body, in use, may be arranged to expose a portion of the trigger. Thus, the relative movement of the housing portions may provide a visual indication that the trigger is ready.

The force required to enable the trigger to activate the drive mechanism may be selected such that the drive mechanism may only be released when the forward end of the injection device (for example the front end of the forward housing) is in contact with an injection site. Without the forward end of the housing in contact with an injection site no (or minimal) reaction force is available for forward pressure on the trigger to act against. Thus, it is not possible to transmit sufficient force to enable the trigger to release the latch and/or to disengage the trigger from its initial resilient engagement (for example with a feature of, or associated with, the forward body).

The injection device may further comprise a cap. The cap may be arranged to close the forward end of the injector housing. The cap may be arranged to be removed prior to use by the user. Prior to removal the cap may obstructs forward movement of the rearward housing relative to the forward housing.

According to a further aspect of the invention there is provided a method of assembling an injection device, comprising:
providing a syringe containing medicament, a housing comprising a forward body and a rearward body, a drive mechanism and a trigger;
inserting the syringe, drive mechanism and forward housing into a forward end of the rearward housing; and
inserting the trigger into a rear end of the rearward housing.

The method may comprise providing a syringe containing medicament, a housing comprising a forward body and a rearward body (which may be axially movably connected when the injection device is assembled), a drive mechanism and a trigger; inserting the syringe, drive mechanism and forward housing into a forward end of the rearward housing; and inserting the trigger into a rear end of the rearward housing.

The method may further comprise forming a forward sub-assembly comprising at least the forward housing. The method may also comprise forming a rearward sub-assembly comprising at least the rearward assembly prior to assembling the injection device. For example, the forward sub-assembly may comprises components which are forward of the syringe and the rearward sub-assembly comprises components which are rearward of the syringe. The syringe may be placed between the forward and rearward sub-assemblies during assembly of the injection device.

The rearward sub-assembly may include a latch (and the trigger may, for example, resiliently engage the latch). The forward sub-assembly may resiliently engage the latch.

The forward body and rearward body may be axially moveable to provide an interlock arrangement. The method may further comprise temporarily preventing operation of the interlock during assembly.

According to a further aspect of the present invention provides an injection device for delivering a dose of medicament from a syringe, the injection device comprising:

a housing comprising a forward body and rearward body and wherein the forward and rearward body are axially moveably interconnected;

a drive mechanism arranged, in use, to be released from an energised position to move a plunger to deliver a dose from said syringe;

a latch arranged to hold the drive mechanism in the energised position; and

a trigger for releasing the latch; wherein

the injection device further comprises an injection complete indication arrangement comprising:
a ribbon spring having a forward end coupled to the plunger and arranged to move with the plunger during activation,
the rear end of the ribbon spring being initially retained on a feature at the rear of the injection device, rearwardly of the latch, such that forward movement of the first end acts to unravel or unwind the ribbon spring; and wherein
the length of the ribbon spring is such that at a pre-determined point in the injection sequence the rear end of the ribbon passes beyond the latch member releasing the ribbon spring to return to a coiled or wrapped configuration to provide an audible and/or tactile indication.

For example, upon recoiling the ribbon spring may strike an inner surface of the latch, drive spring, or other percussive surface within the injection device. The applicants have found that one convenient percussive surface may be part of an intermediate drive member provided as part of the drive mechanism.

In such an arrangement it is important that any unintended audible and/or tactile emissions from the ribbon spring are reduced or prevented. Accordingly, the latch may be provided with a textured rearward face which may provide a cushioning and/or gripping effect between the latch and the ribbon spring. For example the rearward face may be overmoulded with a different material. Alternatively the latch may be formed by a co-injection moulding (also known as twin shot moulding) process which allows different materials to be moulded for a single component.

The ribbon spring may be initially coiled around a surface or member associated with the trigger. Depending upon the particular configuration of the injection device (for example depending upon the space available) the ribbon spring could alternative be coiled around a surface or member associated with the latch. The surface may have a profile which is arranged to substantially match the shape of the ribbon spring coil.

The trigger may include an internal member arranged to interact with the ribbon spring. The internal member may extend substantially transversely across the trigger. The member may have a chamfered side wall to provide an angled running surface for the ribbon spring.

Whilst the invention has been described above, it extends to any inventive combination of the features set out above, or in the following description or drawings.

### BRIEF DESCRIPTION OF THE DRAWINGS

The invention may be performed in various ways, and an embodiment thereof will now be described by way of example only, reference being made to the accompanying drawings, in which:

FIG. 1 is an exploded view of an autoinjector according to a first embodiment showing the syringe, forward sub-assembly and rearward subassembly;

FIG. 2 is an exploded view of the forward sub-assembly of FIG. 1;

FIG. 3 is an exploded view of the rearward sub-assembly of FIG. 1;

FIG. 4 shows two cross-sectional views of the first embodiment in a pre-use configuration;

FIG. 5 shows two cross-sectional views of the first embodiment in a ready configuration (in which the injector has been pressed against an injection site);

FIG. 6 shows two cross-sectional views of the first embodiment in an injection complete configuration (prior to removal of the device from the injection site);

FIG. 7 shows two cross-sectional views of the first embodiment in a post-delivery configuration (after removal of the device from the injection site);

FIG. 8, shows partial cross-sectional views of alternate embodiments;

FIG. 9, shows an exploded partial cross-sectional view of an indicator arrangement for use in an embodiment;

FIG. 10, shows a partial cross-section showing the operational sequence of the indicator arrangement of FIG. 9;

FIG. 11, shows a partial cross-section of a trigger button for use in an embodiment; and

FIG. 12, shows a view of an alternate trigger button for use in an embodiment.

### DETAILED DESCRIPTION OF EMBODIMENTS

Front as used herein will be understood to refer to the end of the injector assembly (or components thereof) which, in use, are closest to the delivery needle delivery end of the injector (i.e. the end which is pointed at the skin). Rear as used herein will be understood to refer to the end of the pen injector assembly (or components thereof) which, in use, are furthest from the needle delivery end of the injector (i.e. the end which is pointed away from the skin). Forward and rearward will, likewise, be understood to refer to the directions orientated towards the front and rear of the injector assembly.

Axial, radial and circumferential are used herein to conveniently refer to the general directions relative to the longitudinal direction of the injection device (or components thereof). The skilled person will, however, appreciated that these terms are not intended to be narrowly interpreted (and for example, the injection device may have a non-circular and/or irregular form). Typically, regardless of the chosen injector device external profile the cartridge or syringe will have a conventional generally cylindrical form and, as such, the longitudinal axis of the injection device will substantially coincide with (or be parallel to) the axial direction of the syringe.

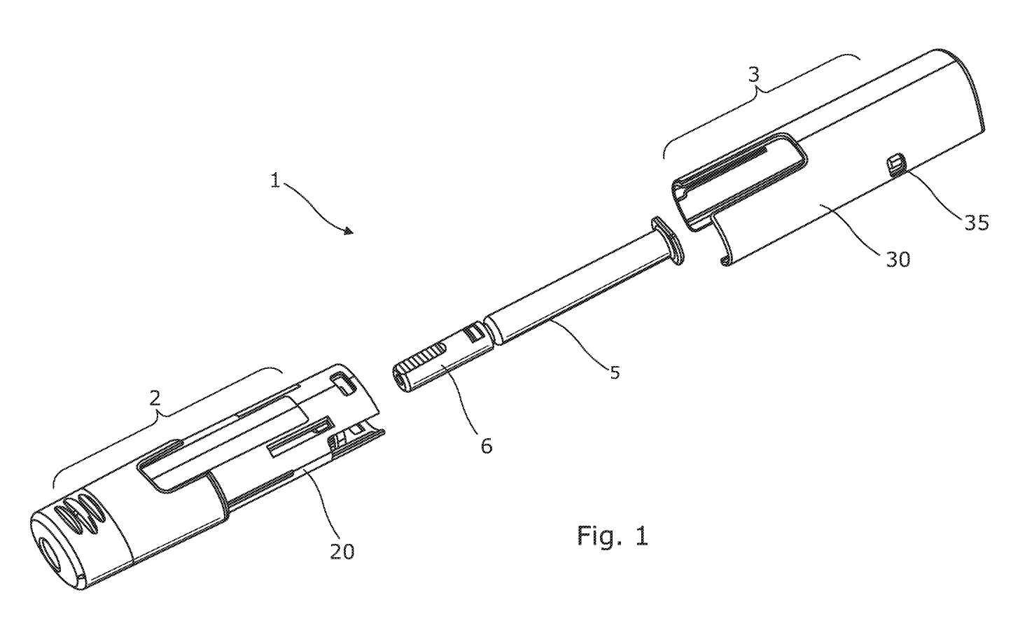

As seen in FIG. 1, an autoinjector 1 in accordance with an embodiment of the invention comprises a housing defined by a forward body 20 and a rearward body 30. A syringe 5 of medicament is provided within the housing. The housing 20, 30 has a generally elongate tubular shape with a generally oval cross-sectional profile.

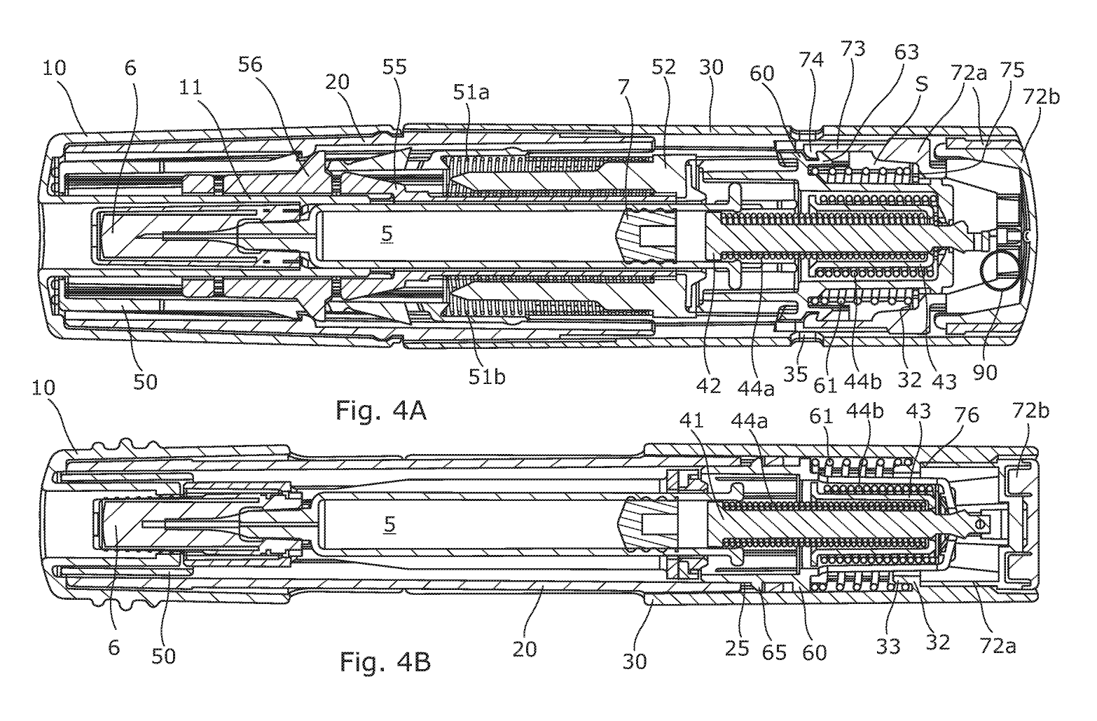

The syringe is a conventional syringe having a bung or stopper 7 within its body (see FIG. 4) and a needle at its forward end which may initially be protected (so as to remain sterile) by a removable needle shield 6. The illustrated autoinjector 1 is generally intended to be a single use device (although the skilled person will appreciate that the invention is not limited to such devices) and, therefore, the exploded view of FIG. 1 may typically represent a final assembly stage in which the syringe is placed into the housing (for example in a manufacturing facility). The end user would typically be provided with the autoinjector 1 preassembled around the syringe 5 (as for example, shown in FIG. 4). As will be explained in further detail below, the autoinjector may conveniently be arranged during manufacture into a forward sub-assembly 2, comprising the forward body 20 and those components which are initially forward of the syringe 5, and a rearward sub-assembly 3, comprising those components which are initially rearward of the syringe 5.

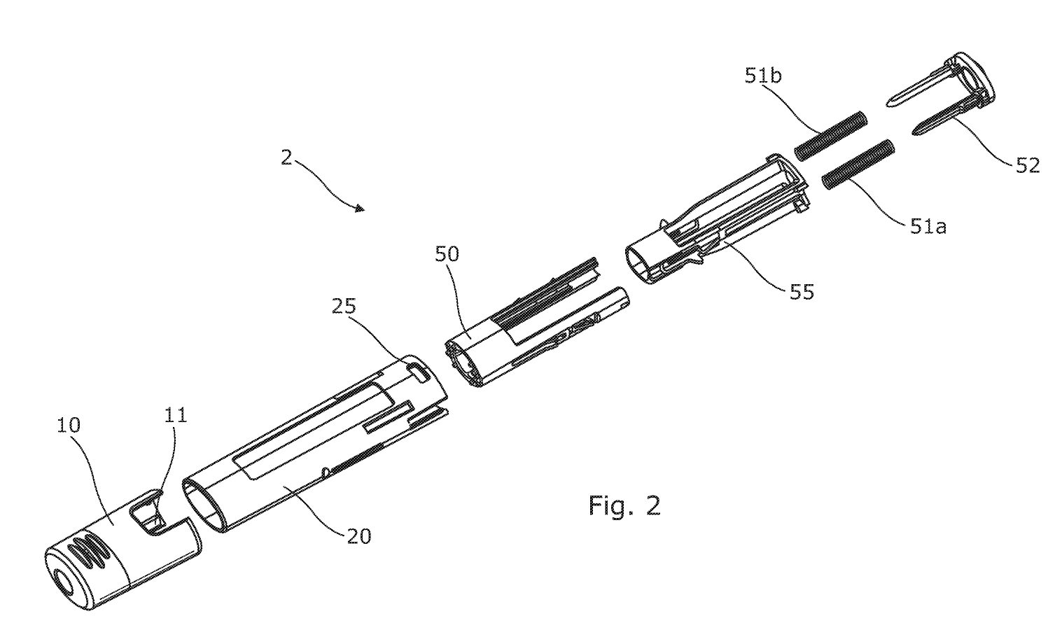

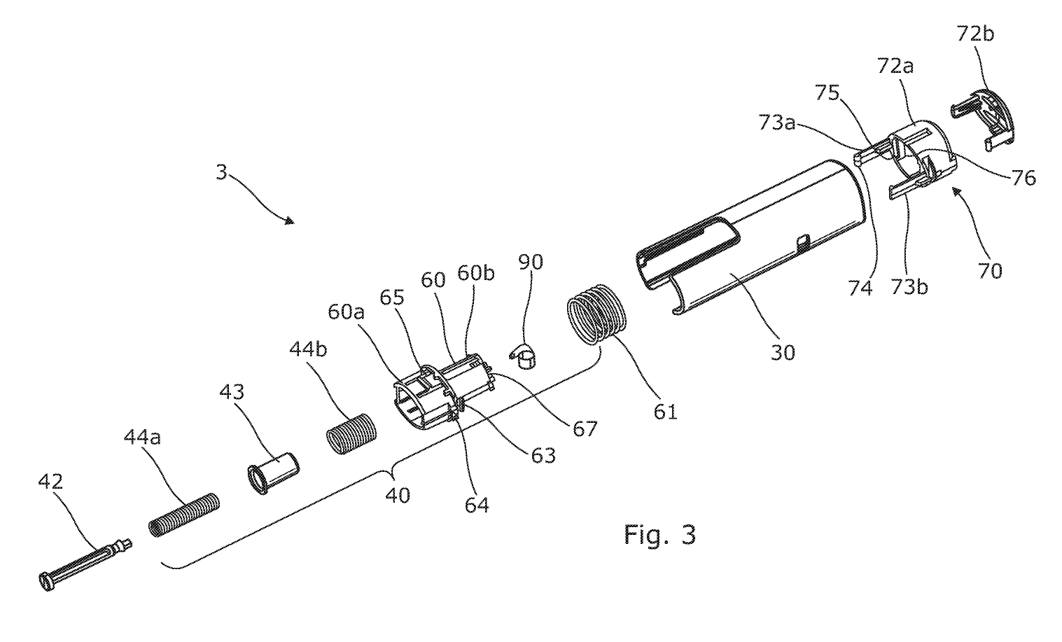

An exploded view of each of the forward and rearward sub-assemblies are shown in FIGS. 2 and 3.

The forward sub-assembly 2 comprises the forward body 20 which is adapted to receive a cap 10 which closes the forward end of the autoinjector 1. The cap 10 may include an internal formation, comprising rearwardly extending members 11, arranged to engage the removable needle shield 6 of the syringe 5 such that removal of the cap 10 from the forward housing 20 during use also removes the removable needle shield 6 from the syringe 5. Within the sub-assembly 2 there is a syringe carrier 55 for movably mounting the syringe within the forward body 20 to enable automatic needle penetration. It may be noted that prior to the removal of the cap 10, the rearwardly extending members 11 of the cap 10 underlie spring fingers 56 of the syringe carrier 55. This arrangement, thus prevents inward movement of the spin fingers 56 prior to removal of the cap 10 and, therefore, blocks unlatching of the syringe carrier 55 and prevents movement relative to the forward body 20.

A needle shroud 50 is also provided and arranged to shroud the needle after use (when the syringe 5 and syringe carrier 55 are in a forward position) to prevent needle stick injuries. The shroud 50 is activated by a pair of side-by-side shroud springs 51a, 51b carried on a spring guide 52. The present application is not limited to any arrangement of the syringe carrier 55 and/or needle shroud 50. As such the operation of the shroud 50 and carrier 55 is not described in detail. However, it may be noted that the arrangement substantially corresponds to the arrangement of the applicants' earlier International Patent Application PCT/GB2011/052557.

The rearward sub assembly 3 includes a trigger button 70 which is inserted into the rearward housing 30 from the rearward end so as to substantially close the rearward end of the injector housing. The trigger button 70 has a cup-like profile with side walls which are arranged to fit within (and be substantially concentric with) the rearward housing 30 and an end wall which closes the rear end of the housing. The trigger button 70 may comprise a single moulded part. However, for ease of manufacture and or assembly, in the illustrated embodiment the trigger button 70 includes a main body 72a and a rear cover 72b. The main body 72a includes a pair of forwardly extending resilient arms 73a and 73b which are arranged to provide an engagement between the trigger button 70 and the injector 1, as will be described in greater detail below.

The rearward sub-assembly 3 also includes the drive mechanism 40, which is inserted into the rearward housing 30 from the forward end. The drive mechanism 40 includes a plunger 42 which is arranged to engage the bung of the syringe in use. The plunger 42 is driven forwards in use by a pair of concentric drive springs 44a and 44b (although it will be appreciated that in other embodiments a single spring may be used) with an intermediate drive member 43 provided therebetween. A latch 60 is arranged concentrically around the drive springs 44, intermediate member 43 and plunger 42. The latch 60 is arranged to hold the plunger 42 against the bias of the springs 44 until the latch is released via the trigger button 70 (when both drive springs and the intermediate drive member are all released together). The latch 60 comprises a rear body portion 60b having a split cylinder profile and defining a latch aperture at its rear end and a forward connecting body portion 60b. The forward body portion 60a has an external profile substantially corresponding to the interior profile of the rearward end of the forward housing 20. The basic functional operation of the drive mechanism 40 is substantially as described, for example, in the applicants' earlier International Patent Applications PCT/GB2011/051950 and PCT/GB2014/052276.

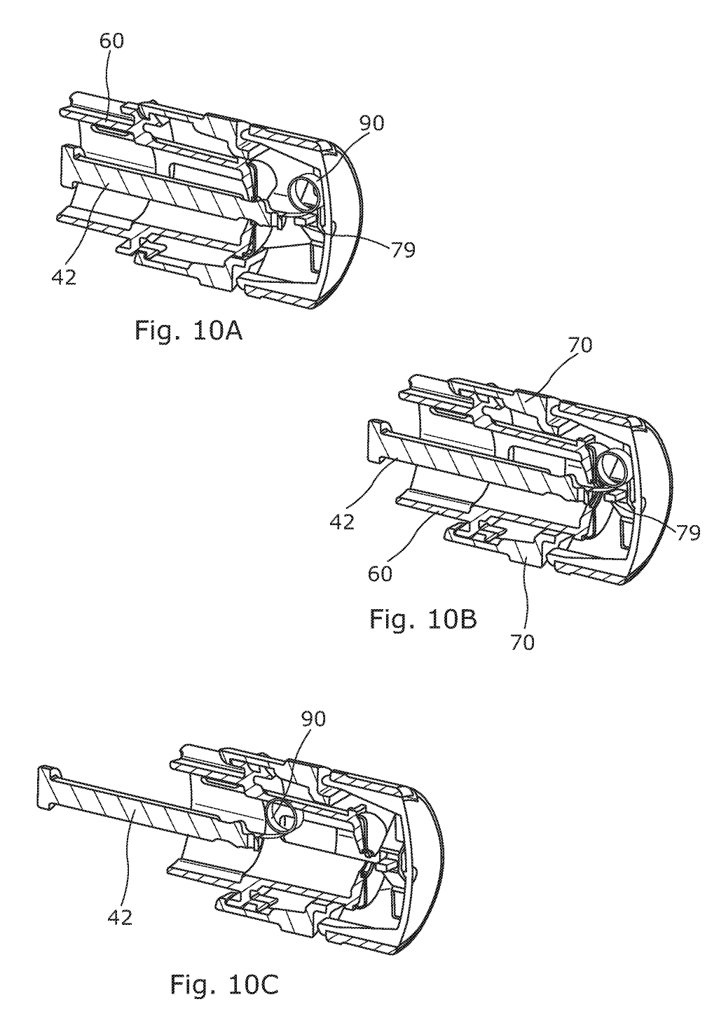

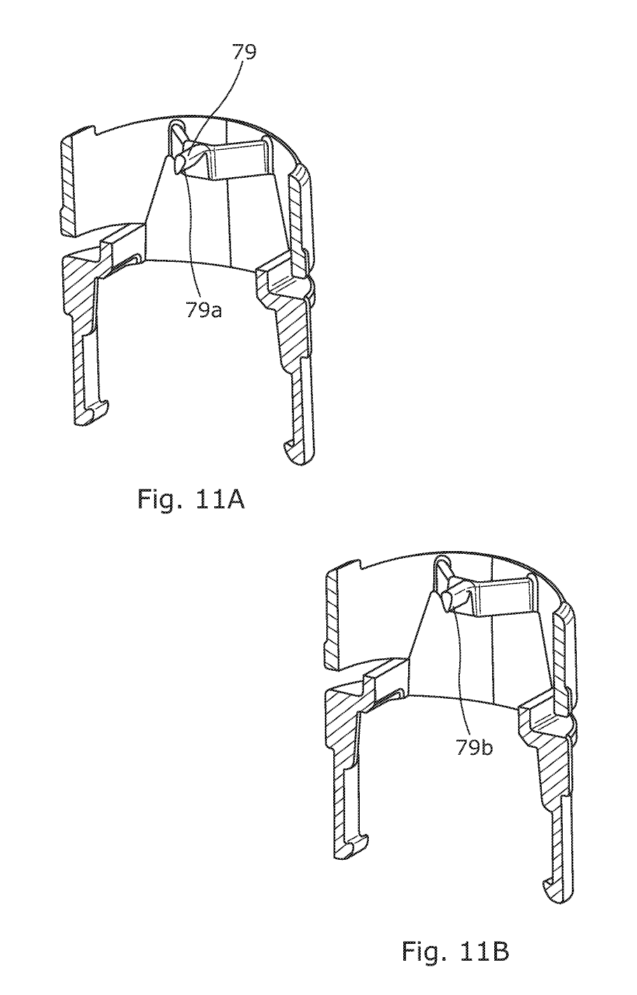

The rearward assembly also includes an indicator arrangement 90 which will be described below with reference to FIGS. 10 and 11.

An interlock spring 61 (the function of which is described below) is provided within the rearward sub-assembly 3 and is captive between the latch 60 and rearward housing 30.

The assembly of the autoinjector according to embodiments of the invention may be particularly convenient since only the trigger button 70 need be inserted from the rearward end of the rearward housing 30. Further, with the components of the drive mechanism 40 inserted from the forward end of the rearward housing 30 the engagement of the latch 60 and trigger button 70 may enable the rearward sub assembly 3 to be initially assembled before being offered up to the forward sub assembly 2. The components of the forward sub-assembly 2 may generally be arranged within the forward body housing 20 and the sub-assemblies snapped together with the syringe in place. All of the components of the forward sub-assembly 2 are therefore also connected to the forward end of the rearward sub-assembly 3. In order to ensure that no unintended activation of the latch member 60 or interlock arrangement occurs during connection of the sub-assemblies 2, 3, at least one aperture 35 may be provided in the rear housing 30 through which operation of the mechanism may be temporarily prevented.

Further details of the autoinjector will now be described with reference to FIGS. 4A and 4B which show a cross section view of the injector in a fully-assembled, pre-use configuration. The two cross-sections are viewed from planes extending through the longitudinal axis of the autoinjector and substantially perpendicular to one another. As the housing of the autoinjector 1 has a substantially oval or elliptical profile it will be noted that FIG. 4A corresponds to a plane which is aligned with the major axis and FIG. 4B corresponds to a plane which is aligned with the minor axis.

In FIG. 4, the forward and rearward sub-assemblies 2, 3 have been connected such that the housing fully encloses the syringe 5. This is the configuration in which the autoinjector may typically be provided to the end-user. The cap 10 is in position and encloses the front end of the forward body 20 of the housing and the trigger button closes the rear end of the rearward body 30 of the housing.

The connection between the sub-assemblies 2, 3 is provided via the latch 60 which it acts as an intermediate connecting member. The interconnection is a resilient snap fit connection which provides for ease of manufacture particularly for a single-use device (which the user will not be required to disassemble). Thus, it may be noted that the forward portion 60a of the latch 60 has been inserted into the rearward end of the forward housing 20. The forward housing 20 and latch 60 are provided with cooperating engagement features 25, 65 to provide an automatic connection when brought together. In the illustrated embodiment the engagement features comprise a cut-out 25 in the forward housing and an outward projection 65 in the forward portion 60a of the latch. It may be noted that in the illustrated embodiment the rearward housing 30 extends over the cut out 25, thereby covering the opening in the housing and preventing the user from disengaging the projection 65 to force separation of the housing body portions 20 and 30.

Within the rear sub-assembly 3, the latch 60 is axially slidably connected to the rearward housing 30 via the trigger button 70. The rearward housing 30 includes a radial flange 32 which extends from the interior bore of the rearward housing 30. It will be noted that the radial flange is spaced radially apart from the inner surface of the rearward housing wall along part of its circumference as shown in FIG. 4A (whereas in the cross section of 4B the flange includes connecting portions extending from the rearward housing). The resilient arms 73 of the trigger button 70 pass between the flange 32 and the inner side wall of the rearward housing 30 to engage the latch 60 (in other words the arms of the trigger button 70 pass over the flange 32 before engaging the latch 60). The forward ends of the resilient arms 73 are provided with inwardly projecting tabs which cooperate with corresponding outwardly projecting tabs 63 provided on a radially outer portion of the latch 60. It will be noted that both the tabs 63 and 74 have an angled profile such that (as will be explained below) the resilient arms 73 may disengage and be deflected outwardly to allow forward movement of the trigger button 70 relative to the latch 60 but prevent disengagement in the reverse direction.

Thus, it will be noted that an axially extending (and at least partially annular) space S is defined between interior of the forwardly extending arms 73 of the trigger button 70 and the exterior of the rearward portion 60b of the latch member 60. The radially spaced portion of the flange 32 is captive within the space S. Also captive within the space S is the interlock spring 61. The interlock spring 61 may, thus, act between a forward face of the flange 32 and a rearward face of the latch 60. Each of the latch 60 and flange 32 may define a seat for receiving an end of the interlock spring (the seat may for example comprise an at least partial annular groove). As such, the interlock spring is arranged to bias the flange 32 rearwardly relative to the latch 60 and, as a result, biases the rearward housing 30 rearwardly relative to the forward housing 20.

In the initial position of FIG. 4, the flange 32 acts as a blocking formation preventing forward movement of the trigger button 70. It will be noted that both a forward facing inner shoulder 75 and a forward facing end wall 76 of the button form forward surfaces which engage a corresponding rearward facing surface of the flange 32. Thus, in the initial position the trigger button 70 is not free to move relative to the housing of the autoinjector and as such the flange 32 and rearward housing 30 provide an interlock arrangement.

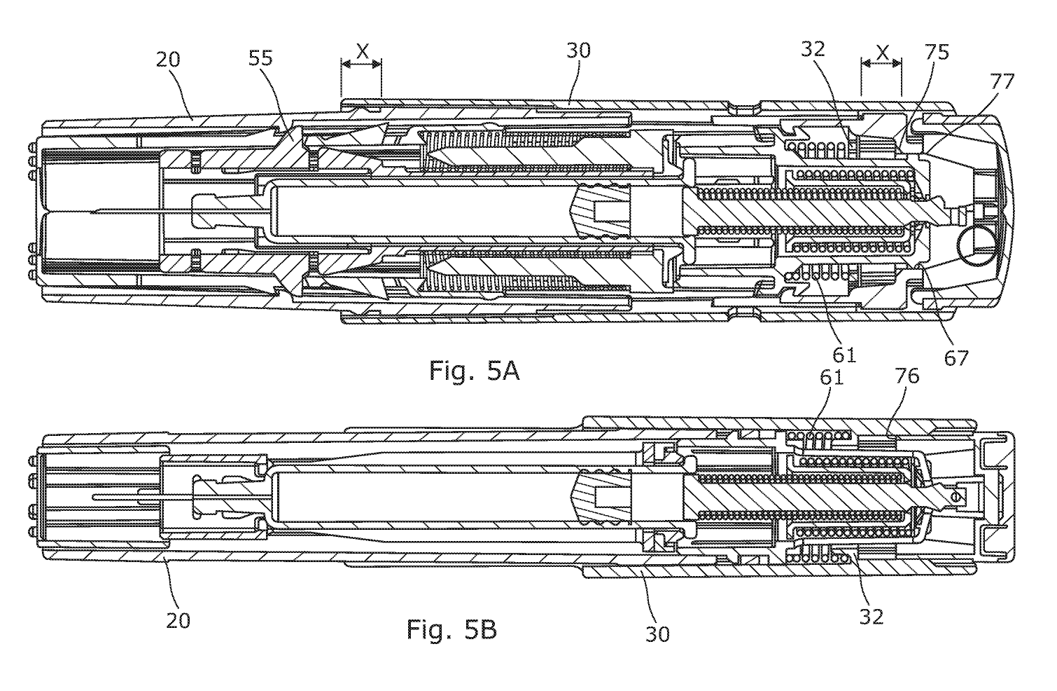

The firing sequence of the autoinjector according to the first embodiment will now be described with reference to FIGS. 5 to 7 which correspond to the cross sections of FIG. 4 in various stages of operation.

After removal of the cap 10 (which also removes the removable needle shield 6) the user places the forward end of the autoinjector 1 against the injection site. The removal of the cap 10 enables the forward 20 and rearward 30 housing bodies to slide relative to one another (as previously the rearward end of the cap abutted the forward end of the rearward housing). After removal of the cap the device is ready to be fired but the interlock provided by the flange 32 will initially be held in place by the interlock spring 61. Further it may be noted that as the user will grasp the rearward housing 30 and the trigger button 70 is blocked by the flange 32 of the housing any force applied to the button 70 prior to positioning the autoinjector against an injection site will only be reacted through the rear housing. In order for the trigger 70 to either be released from its engaged position on the latch 60 and release the latch 60 a reaction force must be transmitted through the injection site to the forward body 20 (or component associated with the forward body).

Once the user presses the device against the skin the rearward body housing 30 is urged forwardly relative to the forward body housing 20, by a distance X, as shown in FIG. 5. The reaction force through the forward body 20 allows the rearward body 30 to slides forward carrying with it the flange 32 (which therefore moves relative to the latch 60) to compresses the interlock spring 61. This forward movement means that the blocking formation provided by the flange 32 is moved forward relative to the trigger button 70. Thus the flange is spaced forwardly apart from the both the shoulder 75 and forward edge 76 of the trigger button 70, by the same axial distance X. As the trigger button 70 is coupled to the latch 60, it remains in a fixed axial position relative to the forward housing 20. Thus, the trigger button 70 is partially revealed at the rear of the rearward housing 30 due to the relative forward movement of the rearward housing. It may be noted that in this position the outer surface of the rear portion 60b of the latch 60 remains radially adjacent to at least a portion 77 of the inner wall of the trigger button. In particular a plurality of radially outwardly projecting bosses 67 of the latch 60 are in abutment with the inner wall section 77 of the trigger button 70. Thus, the rear portion 60b of the latch 60 is unable to deflect radially outwardly to release the plunger 42 to move under the bias of the drive spring 44.

With the interlocking arrangement moved to an unlocked position the user may now depress the trigger 70 in a forward direction. However, it is worth noting that in this particular embodiment the user's sequence of operation is not forced. In other words the user may either press the device against the injection site and then depress the trigger button or may alternatively hold the trigger button whilst pressing the device against the injection site (it is only a requirement that both activation steps are performed).

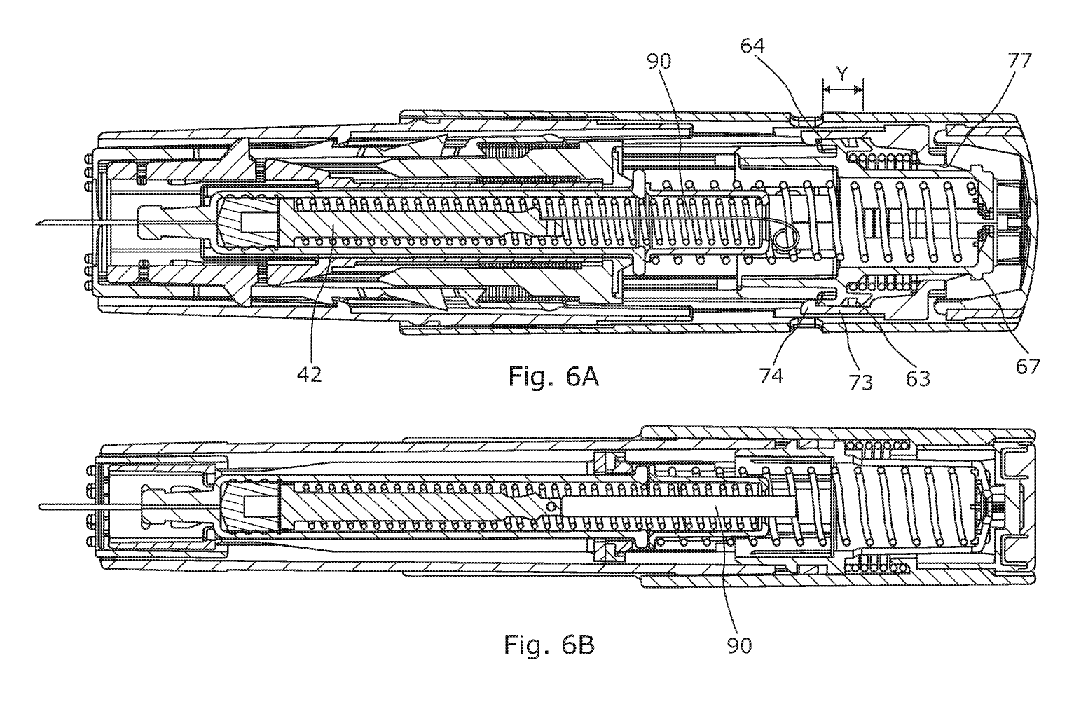

As shown in FIG. 6, the trigger button 70 is displaced forwardly, by a distance Y, relative to the latch 60. The force applied to the trigger button is reacted through the forward housing 20 and the latch 60 to allow the resilient arms 73 to deflect outwardly disengaging the tab 74 from the corresponding tab 63 of the latch and allowing the trigger to move forward. As the trigger moves forward the bosses 67 of the latch 60 move axially clear of the walls 77 on the interior of the trigger button 70. Thus the split cylinder profile of the rearward portion 60b of the latch 60 may deflect radially outwardly to release then end of the plunger 42 to move forward under the force of the drive springs 44a and 44b. Due to the initial stiction between the bung 7 and syringe 5 and/or the substantial incompressibility of the medicament initial movement of the plunger acts to move the syringe 5 and syringe carrier 55 forwardly to cause the needle of the syringe to penetrate the skin. Once the syringe reaches its forwardmost position the syringe carrier 55 reaches a stop position and continued movement of the plunger serves to dispense the medicament.

It may be noted that the forward movement of the trigger button 70 causes the tabs 74 of the resilient arms 73 to ride over an overhang 64 formed forward of the tabs 63 on the latch 70. Thus, the resilient arms 73 may be arranged to cause the tabs 74 to snap into engagement with the latch 60 in a second, axially forward position after activation. This both serves to retain the engagement between the rearward housing 30 and forward housing 20 (via the latch 60 and trigger 70 trapping the flange 32) and to ensure that the trigger button 70 remains in a forward position to indicate that the device has been used and cannot be re-fired. In this arrangement the forward body 20 and rearward body 30 of the housing may no longer be slidably engaged (rather they are axially fixed as the interlock spring 61 is fully compressed).

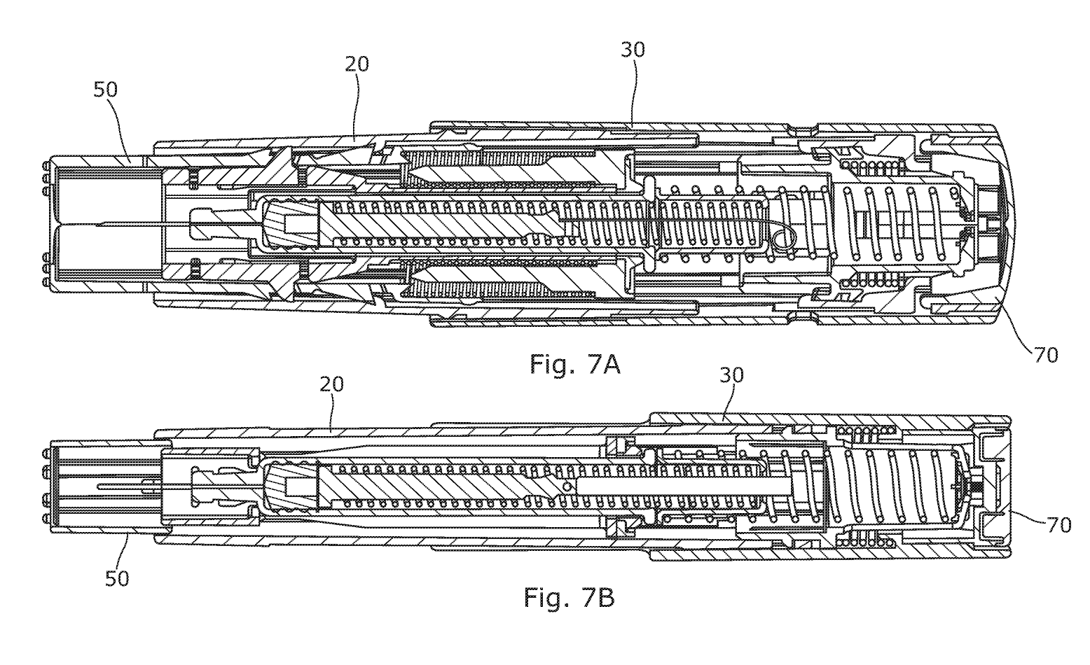

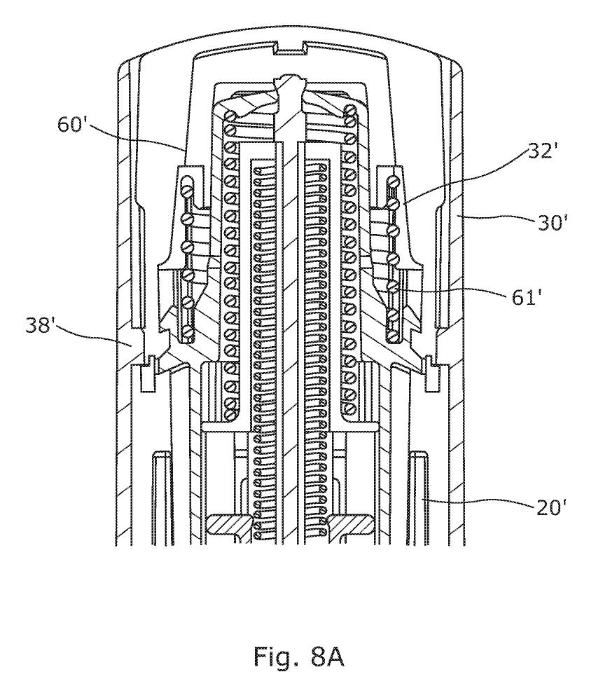

During the activation movement of the syringe carrier 55, the needle shroud 50 is released for forward movement relative to the forward housing 20 under the biasing of the shroud springs 51a, 51b. Thus, as shown in FIG. 7, upon removal of the autoinjector from the injection site the needle shroud 50 is urged forward to automatically shroud the needle of the syringe 5 (which remains in the forward position). The shroud 50 is provided with latch features which lock the shroud in its fully forward position relative to the housing.

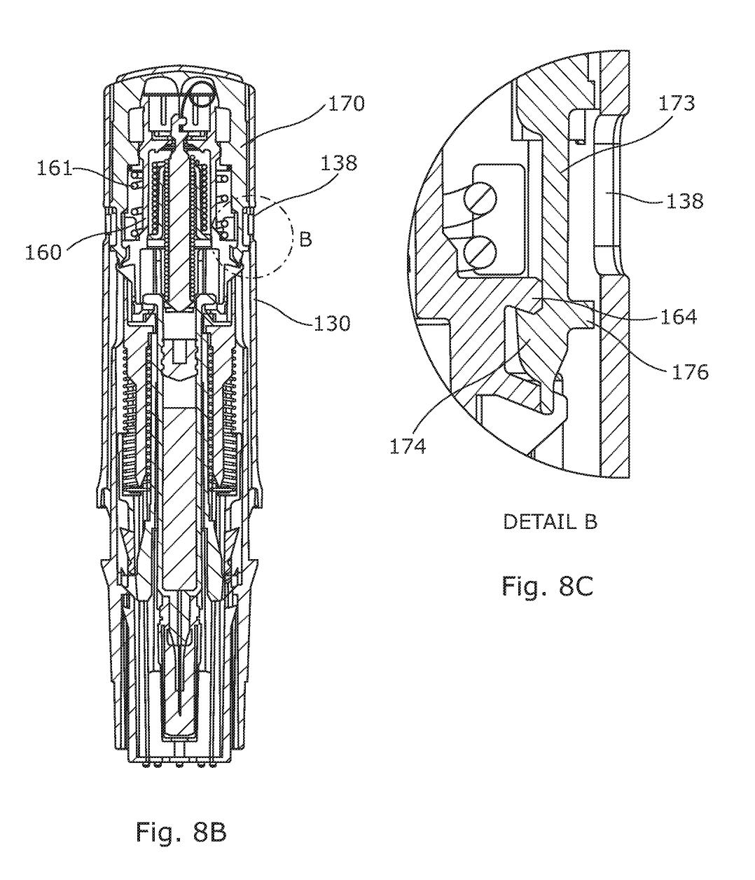

FIG. 8A illustrates the latch 60 and interlock arrangement according to an alternative embodiment. This embodiment works in the same manner as the first embodiment but includes additional optional features. In this embodiment the rearward body 30′ is provided with a profiled inner wall arranged to block radial deflection of the resilient legs of the trigger 70′ until the rearward body 30′ has moved forward to unlock the interlock. The inner wall includes radially inwardly extending abutments 38′ which are aligned with the tabs 74′ of the trigger 70′ so positively block disengagement between the trigger 70′ and the latch 60′ until the rearward body 30′ has moved forward against the interlock spring 61′. In contrast to the first embodiment, this embodiment ensures that the activation sequence must require the device 1 be pressed against the injection site before the trigger 70′ is depressed (in order to ensure that the blocking abutments 38′ clear the trigger 70′).

FIGS. 8B and 8C illustrates an alternate arrangement which operates in a similar manner to that of FIG. 8A. The injector device of this embodiment includes a latch 160 and a trigger button 170 substantially as described above. The forward arms 173 of the trigger button 170 extend forwardly to engage the tabs 164. In the pre-use position shown in the figure an external outwardly radially extending boss 176 abuts the inner surface of the rear body housing 130 and prevents outward deflection of the leg 174. Thus, the button 170 cannot be urged forwardly to release the latch 160.

The housing 130 is provided with an aperture 138 rearwardly of the boss 176. It will be appreciated that when the rear housing 130 moves forward as a result of the device being pressed against an injection site the aperture 138 moves into alignment with the boss 176 on the arm 173. After the device has been presented to the injection site and the rear body 130 urged forwardly, the arm 173 is free to move radially outwardly and allows the button 170 to be depressed. Accordingly, the device of FIG. 8B may only be activated by sequentially pressing the device against the injection site prior to depressing th injection button 170.

The aperture 138 could alternatively be a recess but an aperture 138 may advantageously provide a visual indication of activation of the device by virtue of the bosses 176 being visible through the aperture 138 when they move into alignment. As such, the user is provided with an indication that the device is ready to be fired by means of the button 170.

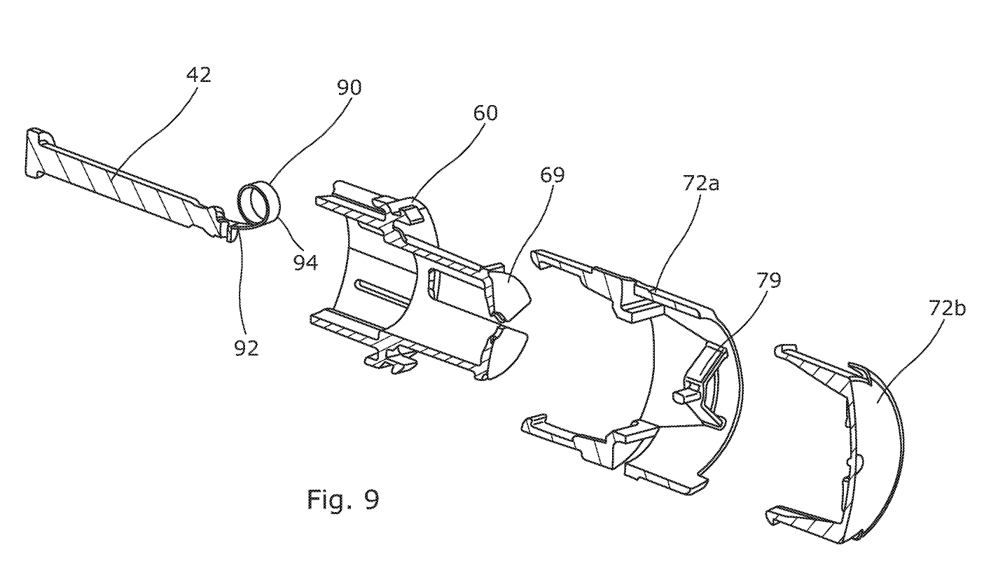

FIG. 9 shows an exploded cross sectional view of an audible indication arrangement for use in embodiments. The indicator is typically arranged to provide an audible (and optionally tactile) indication which may be in the form of a “click”. The indication may be arranged to be triggered as the plunger 42 reaches a fully forward position to provide the user with an indication that a full dose of medicament has been delivered and, therefore, that the autoinjector may be removed from the injection site. The indicator comprises a flexible member in the form of a ribbon spring 90 (which may be a constant force spring). A forward end 92 of the ribbon is attached to the rearward end of the plunger 42 and the rearward portion 94 of the ribbon is in the form of a coil.

As best seen in FIG. 10A, which shows the unfired configuration, indicator 90 and end of the plunger 42 are initially positioned rearwardly of the latch member 60. As the trigger is used to activate the drive mechanism (FIG. 10B) the plunger 42 is released from the latch 60 and moves forwardly. It may be noted that during activation the transverse member 79 which extends across the rear of the trigger button 70 may engage the end of the plunger 42 to help urge it forwards through the aperture of the latch. In other embodiments this function may be provided by a boss projecting from the rearward inner surface of the trigger button (see for example FIG. 8). The provision of the transverse member 79 enables additional space to be provided between the rearward surface of the latch 60 and the forward surface of the end of the trigger 70 to accommodate the coil 94.

After release of the drive mechanism the plunger 42 moves forward within the autoinjector relative to the latch 60 and draws with it the forward end 92 of the indicator. The coil 94 at the rear of the indicator does not fit through the aperture of the latch 60 (and additionally may be wrapped around the transverse member 79) so is not carried forwardly but unravels. At, or typically slight prior to, the plunger 42 reaching its forwardmost position the indicator 90 will reach its fully unravelled arrangement. Thus, the rearwardmost end of the indicator will pass through the aperture of the latch 60. Once the rearward end of the indicator is forward of the rearward portion of the latch 60, the indicator will recoil due to its resilience. Thus, as shown in FIG. 10C the indicator will spring forwards towards the end of the plunger 42 and will strike a convenient surface of the autoinjector (which may for example be an inner surface of the latch, the inside of the drive spring or an intermediate drive member) to provide an audible click. In the illustrated embodiment the surface against which the coil 94 strikes is the intermediate drive member 43 (for example the rearward facing end surface of the intermediate member 43).

In order to increase the effectiveness of audible indications it is desirable to eliminate or minimise any intermediate noises other than the final release/striking of the indicator 90. One means of reducing such noises is to provide a suitable surface to the rear of the latch member over which the indicator 90 will travel. For example, an overmoulding or comoulded portion 69 may be provided on the rear surface. The overmoulded or comoulded portion 69 may use a material which is specifically chosen for acoustic reasons (whilst the material of the latch itself may be primarily defined by its function). For example a material for the portion 69 may have different shore hardness to the material of the main body of the latch 60. For example, an appropriate shore hardness may be selected to cushion any unwanted contacts and prevent unwanted “clicks”.

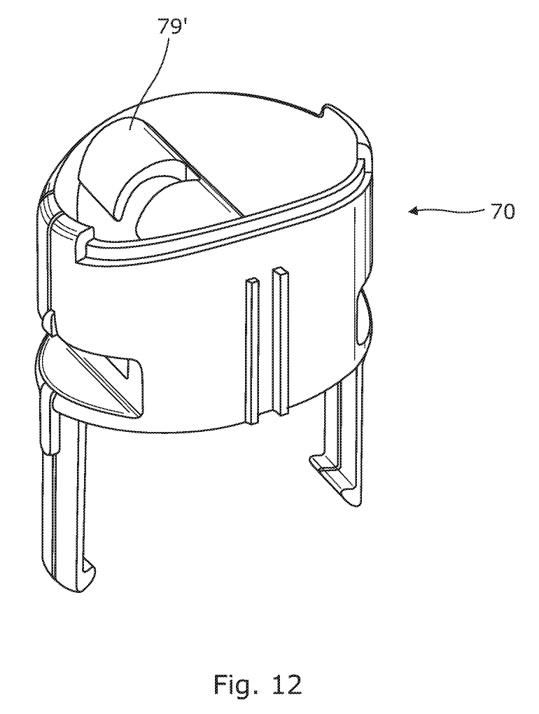

Further, as shown in FIG. 11 the transverse member may be provided with at least one chamfered side wall 79a, 79b to provide a smooth running surface for the indicator 90. The chamfer may be single sided 79a or double sided 79b. Alternatively, as shown in FIG. 12 the transverse member 79′ may have a profile which is intended to substantially match the curvature of the coil of the indicator 90. Thus, the rear section may 94 of the indicator may be coiled around the member 79′ and have a smooth running surface across the member.

Although the invention has been described above with reference to a preferred embodiments, it will be appreciated that various changes or modifications may be made without departing from the scope of the invention as defined in the appended claims.

1. An injection device for delivering a dose of medicament from a syringe, the injection device comprising:
a housing comprising a forward body and rearward body and wherein the forward and rearward body are axially moveably interconnected;
a drive mechanism arranged, in use, to be released from an energised position to deliver a dose from said syringe;
a latch arranged to hold the drive mechanism in the energised position; and
a trigger for releasing the latch; wherein
the injection device further comprises an interlock arrangement moveable between a locked position in which release of the drive mechanism is prevented and an unlocked position in which the drive mechanism may be released, the interlock arrangement comprising:
an internal blocking formation associated with the rearward body and positioned between the latch and trigger, and wherein
in use, forward movement of the rearward body relative to the forward body moves the blocking formation into the unlocked position.

2. An injection device as claimed in claim 1, wherein, the trigger and latch remain relatively axially fixed when the interlock is moved to the unlocked position.

3. An injection device as claimed in claim 1, wherein, in the locked position, the blocking formation blocks forward movement of the trigger.

4. An injection device as claimed in claim 1, wherein the blocking formation comprises a rearwardly facing surface for cooperating with a forwardly facing surface of the trigger when in the locked position.

5. An injection device as claimed in claim 1, wherein trigger is disposed at a rearward end of the housing and arranged for forward movement relative to the housing to release the latch and wherein the trigger extends forwardly within the interior of the rearward body and the blocking formation is at least partially within the interior of the trigger.

6. An injection device as claimed in claim 1, wherein the latch is at least partially enclosed by the trigger.

7. An injection device as claimed in claim 5 wherein the trigger extends forward to engage a feature of, or associated with, the forward body to retain the forward and rearward bodies in slidable engagement.

8. An injection device for delivering a dose of medicament from a syringe, the injection device comprising:
a housing comprising a forward body and rearward body and wherein the forward and rearward body are axially moveably interconnected;
a drive mechanism arranged, in use, to be released from an energised position to deliver a dose from said syringe;
a trigger disposed at a rearward end of the housing for releasing the drive mechanism; wherein
trigger extends forwardly within the interior of the rearward body and over at least one feature of, or associated, with the rearward body and engages a feature of, or associated with, the forward body to retain the forward and rearward bodies in slidable engagement.

9. An injection device as claimed in claim 8, wherein at least one feature of, or associated, with the rearward body comprises a blocking formation of an interlock arrangement.

10. An injection device as claimed in claim 8, wherein the trigger comprises a cup-shaped button having a rearward end which closes the rear end of the housing and side portions which extend forwardly within the interior of the rearward body.

11. An injection device as claimed in claim 7, wherein the trigger is arranged to have a snap fit engagement with the feature of, or associated with the forward body.

12. An injection device as claimed in claim 11, wherein the trigger includes a resilient catch for engaging the feature of, or associated with the forward body, and arranged to disengage to allow forward movement of the trigger.

13. An injection device as claimed in claim 12, wherein the resilient catch is arranged to re-engage a feature of, or associated with the forward body upon reaching an activation position.

14. An injection device as claimed in claim 12, wherein the rearward body is arranged to prevent disengagement of the resilient latch until the rearward body is moved forward relative to the forward body.

15. An injection device as claimed in claim 14, wherein the inner surface of the rearward body is profiled such that the relative forward movement of the rearward body creates clearance between the resilient member and the rearward body to allow disengagement of the resilient member.

16. An injection device as claimed in claim 7, wherein the trigger engages a feature of the latch.

17. An injector as claimed in claim 1, wherein the drive mechanism further comprises a plunger arranged for expelling medicament from the syringe and wherein the latch engages a rearward portion of the plunger to hold the drive mechanism in the energised position.

18. An injection device as claimed in claim 1, wherein the blocking formation is radially spaced from the inner surface of the rearward body.

19. An injection device as claimed in claim 18, wherein the blocking formation comprises an internal wall extending across at least a portion of the rearward body.

20. An injection device as claimed in claim 19, wherein at least a portion of the internal wall extends generally axially relative to the rearward body.

21. An injection device as claimed in claim 18, wherein the blocking formation is at least substantially or partially annular.

22. An injection device as claimed in claim 18, wherein the blocking formation is generally concentric with the rearward body.

23. An injection device as claimed in claim 1, wherein the latch is connected to the forward body.

24. An injection device for delivering a dose of medicament from a syringe, the injection device comprising:
a housing comprising a forward body and rearward body and wherein the forward and rearward body are axially moveably interconnected;
a drive mechanism arranged, in use, to be released from an energised position to deliver a dose from said syringe;
a latch arranged to hold the drive mechanism in the energised position; and
a trigger for releasing the latch; wherein
the latch is arranged to provide an interconnecting member between the forward body and rearward body.

25. An injection device as claimed in claim 24, wherein the latch engages the forward body and the trigger engages the latch such that a feature of, or associated with, the rearward body is restrained between the latch and trigger.

26. An injection device as claimed in claim 1, wherein the rearward body is biased rearwardly relative to the forward body.

27. An injection device as claimed in claim 26, wherein the rearward and forward bodies are biased by a spring acting between the latch and locking formation.

28. An injection device as claimed in claim 27, wherein the latch comprises a rearwardly facing surface for engaging a first end of the spring and the blocking formation comprises a forwardly facing surface for engaging a second end of the spring.

29. An injection device as claimed in claim 1, wherein the rearward body is arranged to be held by the user during use and to move forwardly relative to the forward body when the injection device is pressed against an injection site.

30. An injection device as claimed in claim 1, wherein forward movement of the rearward body, in use, is arranged to expose a portion of the trigger so as to provide a visual indication that the trigger is ready.

31. An injection device as claimed in claim 1, wherein the force required to enable the trigger to activate the drive mechanism is selected such that the drive mechanism may only be released when the forward end of the injection device is in contact with an injection site to provide a reaction force.

32. An injection device as claimed in claim 31, wherein the reaction force from the forward end of the injection device is required to overcome a resilient engagement of the trigger and/or to release a latch member.

33. An injection device as claimed in claim 1, further comprising a cap, closing the forward end of the injector housing and arranged to be removed prior to use by the user, wherein the cap obstructs forward movement of the rearward housing relative to the forward housing prior to removal.

34. A method of assembling an injection device, comprising:
providing a syringe containing medicament, a housing comprising a forward body and a rearward body, a drive mechanism and a trigger;
inserting the syringe, drive mechanism and forward housing into a forward end of the rearward housing; and
inserting the trigger into a rear end of the rearward housing.

35. A method of assembly as claimed in claim 34, wherein the method further comprises forming a forward sub-assembly comprising at least the forward housing and a rearward sub-assembly comprising at least the rearward assembly prior to assembling the injection device.

36. A method of assembly as claimed in claim 35, wherein the forward sub-assembly comprises components which are forward of the syringe and the rearward sub-assembly comprises components which are rearward of the syringe.

37. A method of assembly as claimed in claim 36, wherein the syringe is placed between the forward and rearward sub-assemblies during assembly of the injection device.

38. A method of assembly as claimed in claim 34, wherein the rearward sub-assembly includes a latch and the trigger resiliently engages the latch.

39. A method of assembly as claimed in claim 38, wherein the forward sub-assembly resiliently engages the latch.

40. A method of assembly as claimed in claim 34, wherein the forward body and rearward body are axially moveable to provide an interlock arrangement and the method further comprises temporarily preventing operation of the interlock during assembly.
